(19) United States
(12) Patent Application Publication
(10) Pub. No.: US 2024/0347207 A1
(43) Pub. Date: Oct. 17, 2024

(54) METHODS AND SYSTEMS FOR PREDICTING THE RISK OF METASTASIS USING MULTI-MODALITY DATA

(71) Applicant: AIRAMATRIX PRIVATE LIMITED, Thane-West (IN)

(72) Inventors: Nitin SINGHAL, Thane (IN); Aditya VARTAK, Thane West (IN); Nilanjan CHATTOPADHYAY, Mumbai (IN)

(73) Assignee: AIRAMATRIX PRIVATE LIMITED, Thane-West (IN)

(21) Appl. No.: 18/631,898

(22) Filed: Apr. 10, 2024

(30) Foreign Application Priority Data
Apr. 12, 2023 (IN) 202321027227

(51) Int. Cl. G16H 50/30; G16H 30/40; G16H 50/20

(52) U.S. Cl. CPC G16H 50/30; G16H 30/40; G16H 50/20

(57) ABSTRACT

Embodiments herein disclose multi-modality metastasis risk prediction in subjects post radical prostatectomy. Tumour regions in input histopathological images in the subjects post radical prostatectomy is identified using a semantic segmentation network. At least one patch of a pre-defined size is generated from the identified tumour regions. Image compression is performed on at least one patch to reduce dimensionality. Classification of input data is performed to predict risk of metastasis in the subjects post radical prostatectomy. The classification is based on generation of concatenated feature vectors at training stage and an AI score to predict the risk is then generated.

### CROSS REFERENCE TO RELATED APPLICATION

This application is based on and derives the benefit of Indian patent application 202321027227, the contents of which are incorporated herein by reference

### TECHNICAL FIELD

Embodiments disclosed herein relate to analysis of medical data using deep learning models, and more particularly to multi-modality based prediction of risk of metastasis in subjects post radical prostatectomy.

### BACKGROUND

Prostate cancer is one of the most common forms of cancer in men. One in eight men will be diagnosed with prostate cancer at some point in their life, with disease severity and prognosis varying in a broad spectrum. More than 90% of patients with no metastasis are expected to survive for at least five years, while patients with metastatic prostate cancer show a much poorer prognosis. Patients with localized prostate cancer who are at high risk of developing nodal or distant metastases need to be identified and treated. Metastatic prostate cancer is difficult to treat. The standard treatment is Androgen Deprivation Therapy progressing to a castration-resistant phase. Clinical management is complicated by the wide range of disease manifestation and the current method relies on risk-stratifying patients for discovering and treating the most severe lesions.

Localized prostate cancer is commonly treated by radical prostatectomy (RP). Within 10 years, up to one-third of individuals who have undergone RP for clinically organ-confined prostate cancer develop a biochemical recurrence (BCR). Although many patients with BCR have indolent disease, others eventually develop metastases and succumb to prostate cancer and the metastatic disease is associated with a high death rate. Only 20-30% of metastatic cancer patients survive five years following diagnosis, and roughly 85% of cases are characterized by bone metastases. Patient counselling and salvage therapy selection depend on the capacity to distinguish individuals at risk of metastasis and death, from those with a less aggressive form of the disease.

Currently, determining risk of metastasis in prostate cancer patients after radical prostatectomy is a challenge and there are no efficient techniques for risk prediction. Current techniques (such as tumor, nodes, and metastases (TNM) staging, Gleason score grading, D'Amico risk stratification, and the University of California, San Francisco-Cancer of the Prostate Risk Assessment (CAPRA) score) rely heavily on pathology data, (such as primary and secondary Gleason scores), reporting of which is highly subjective and susceptible to inter and intra-observer variation. Current risk stratification methods are fixed and are based on a restricted number of features.

Recently, tissue-derived genomic biomarkers have demonstrated enhanced prognostic efficacy. However, adoption of these tests outside the U.S. has been low due to costs, dependence on specialized laboratory requirements, and processing time.

Currently, there are no efficient AI based techniques for predicting the likelihood of metastasis in subjects post radical prostatectomy.

### OBJECTS

The principal object of embodiments herein is to disclose methods and systems for predicting risk of metastasis of prostate cancer in subjects post radical prostatectomy using a concatenated feature based classification network.

These and other aspects of the embodiments herein will be better appreciated and understood when considered in conjunction with the following description and the accompanying drawings. It should be understood, however, that the following descriptions, while indicating at least one embodiment and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

Embodiments herein are illustrated in the accompanying drawings, through out which like reference letters indicate corresponding parts in the various figures. The embodiments herein will be better understood from the following description with reference to the drawings, in which:

FIG. 1 illustrates a concatenated feature based classification system for predicting risk of metastasis in prostate cancer patients, according to the embodiments herein;

FIG. 2 illustrates the processes of tumour identification and patch, according to embodiments as disclosed herein;

FIG. 3 depicts the image compression process, according to embodiments as disclosed herein;

FIG. 4 illustrates the classification process for predicting the risk of metastasis in subjects post radical prostatectomy, according to embodiments as disclosed herein;

FIG. 5a illustrates a sample report showing the risk of metastasis as determined by the concatenated feature based classification system, according to embodiments as disclosed herein;

FIG. 5b illustrates a graph which depicts the probability of metastatic event at different time intervals, according to embodiments as disclosed herein;

FIG. 6 depicts the process of tumour identification and patch generation, according to the embodiments herein;

FIG. 7 depicts the process of image compression and classification for predicting

the risk of metastasis, according to the embodiments herein; and

FIG. 8 depicts the process of predicting risk of metastasis, according to the embodiments herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

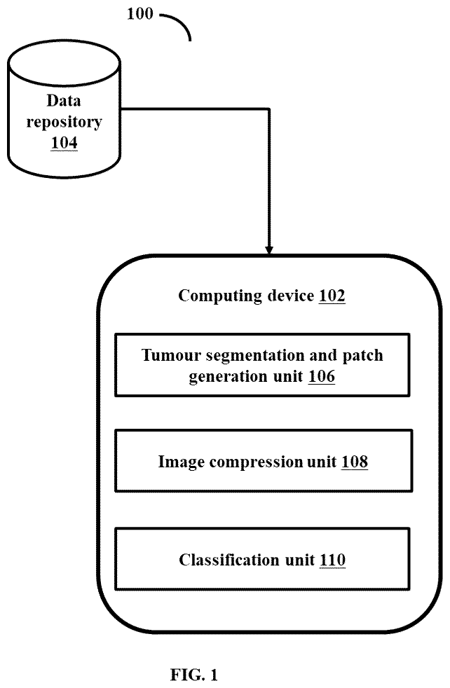

The embodiments herein provides methods and systems for predicting the risk of metastasis in prostate cancer patients. Artificial Intelligence (AI), particularly deep learning-based approaches, employs imaging and categorical clinicopathological data with labels such as “metastasis” or “no metastasis” to train optimized algorithms. AI based approaches can learn from enormous quantities of data across various modalities, such as imaging, molecular markers, and clinicopathologic factors. In contrast to genomic biomarkers, AI systems that exploit digitized images are more affordable and scalable. The embodiments herein employ a multi-modality deep learning system that takes in data from various modalities, such as imaging, molecular markers, and clinicopathologic factors. The deep learning system can learn from enormous quantities of data across various modalities and predict the likelihood of metastases in patients post radical prostatectomy. The embodiments herein enable the early detection of metastasis. Referring now to the drawings, and more particularly to FIGS. 1 through 8, where similar reference characters denote corresponding features consistently throughout the figures, there are shown embodiments.

FIG. 1 illustrates a concatenated feature based classification system 100 for predicting risk of metastasis in subjects post radical prostatectomy, according to the embodiments herein. The system 100 comprises a computing device 102 coupled to a data repository 104. The data repository 104 comprises clinical data, radiology data, molecular markers, and histopathological images. Examples of the data repository 104 can be, but not limited to, a database, cloud storage, memory, and the like. The data repository 104 and the computing device 102 are connected through at least one wireless connection, or a wired connection over a communication network. In an example, the computing device can be located remotely with respect to the data repository 104. The computing device 102 comprises a tumour identification and patch generation unit 106, an image compression unit 108, and a classification unit 110. Each of the units are executed by a processing unit (not shown in FIG. 1).

The images and other data stored in the data repository are input to the tumour identification and patch generation unit 106. The tumour identification and patch generation unit 106 employs a deep learning model that implements a transformer-based architecture for identifying tumour regions. The patch generation unit uses a segmentation network to extract patches (areas) of a pre-defined size having at least 10% tumour from the identified tumour regions. Patches with the highest tumour percentage output by the patch generation unit is then fed to the image compression unit 108 for feature extraction. The image compression unit 108 enables creation of effective training data. The last step is the image classification, where the classification unit 110 performs metastasis classification. At the classification stage, clinical data can also be input to the classification unit 110. The classification unit 110 generates an AI score that depicts the risk of metastasis in a given subject. The various units of the concatenated feature based classification system are explained in detail below.

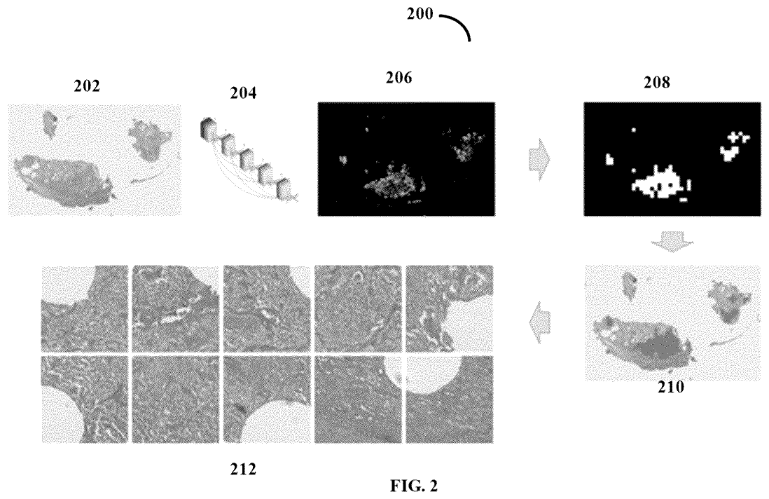

FIG. 2 illustrates the process of tumour identification and patch generation 200, according to the embodiments herein. The first step in the prediction of risk of metastasis in post radical prostatectomy in subjects is identification of tumour from histopathological images 202. The identification of the tumour is performed by a semantic segmentation network 204, such as, but not limited to, a transformer-architecture based SegFormer. It is within the scope of the embodiments herein, to employ other semantic segmentation networks, such as a U-Net.

SegFormer is a deep learning model architecture which has a transformer-based architecture. SegFormer is a semantic segmentation network with a transformer and a MultiLayer Perceptron (MLP) decoder. It employs a hierarchical transformer encoder such as, but not limited to, a Swin transformer, but does not employ positional encoding. Due to the hierarchical nature of the encoder, the network can produce multi-level, multi-scale features with high-resolution coarse features and low-resolution fine-grained features. Lack of positional encoding enables applying the SegFormer model on patches of varying resolutions without degrading performance, which is one of the major limitations of transformer-based networks. The decoder in SegFormer is intended to be lightweight and efficient and simultaneously remaining simple and computationally intensive. SegFormer employs self-attention mechanisms to process the input images and captures long-range dependencies between pixels.

In semantic segmentation, each pixel in the input histopathological images is labelled with a semantic class. The tumour identification and patch generation unit 106 performs the tumour identification by first encoding the input histopathological images using a set of convolutional layers to obtain a set of feature maps. Then, the tumour identification and patch generation unit 106 processes these feature maps using a series of transformer blocks to extract high-level representations that capture both local and global contexts. The tumour identification and patch generation unit 106 transmits the output of the transformer blocks to a decoder network that produces a dense prediction tumour mask 206 for each pixel. SegFormer can capture long-range dependencies between pixels more efficiently, which can be particularly useful for large images or images with complex object interactions. Additionally, the transformer-based architecture used in SegFormer can be trained efficiently on large datasets.

On producing dense prediction tumour masks 206 for each pixel of the input images, the tumour identification and patch generation unit 106 performs patch generation which involves extracting at least one patch having at least 10% tumour 208 from the identified tumour regions. The patch generation is performed by a semantic segmentation network, such as, but not limited to SegFormer. There are different methods to generate patches, such as, but not limited to, sliding window approaches, random sampling, or using pre-defined regions of interest. Patch generation using deep learning models is a common approach in tumour identification and classification.

Patch generation involves extracting small image patches from a larger image, and training a deep learning model, such as SegFormer, to classify the patches as either tumour or non-tumour regions. Patch generation is based on the idea that the visual information in an image is often concentrated in local regions or patches, rather than being distributed evenly across the whole image. Using the patch generation process, the spatial and contextual information in the medical images can be captured at a finer scale than using the whole image. Patch generation improves the accuracy of the tumour detection and localization compared to using the entire image.

In an example herein, the tumour identification and patch generation unit 106 extracts patches of size 256×256×3 having at least 10% tumour 208 from the identified tumour region. The tumour identification and patch generation unit 106 produces top 2×N×N patches 210 with the highest tumour percentage and at least one patch 212 is generated. The top 2×N×N patches 210 with the highest tumour percentage are then chosen by the image compression unit 108 for further processing. However, dimension of the patches can be varied depending on the problem type as the dimension is a hyperparameter whose value is set before the training process begins.

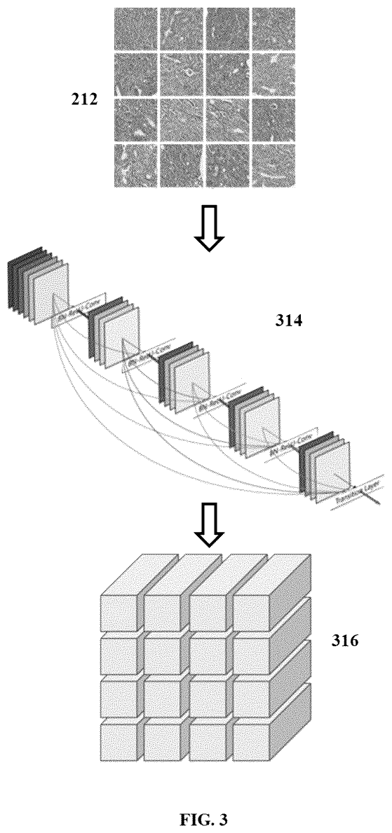

FIG. 3 depicts the image compression process 300, according to the embodiments

herein. On generating the at least one patch 212, the image compression unit 108 preprocesses the patches to normalize their size and intensity values before transmitting the same to the classification unit 110. The image compression unit 108 employs a deep learning model 314, wherein the model is trained utilizing, but not limited to, Diversity Inducing Non-parametric Optimization (DINO) approach and a vast number of existing histopathology data devoid of output labels. DINO is a self-supervised learning method for training deep neural networks to learn useful representations of data. In contrast to traditional supervised learning (which requires labeled data), self-supervised learning aims to learn from unlabeled data by designing pretext tasks that can be used to provide supervision to the network. The DINO method for self-supervised learning involves training a deep neural network to encode images into a set of feature vectors. These feature vectors are then used to cluster similar images together in a high-dimensional embedding space, which is learned by the network. This is achieved through a contrastive learning objective, where the network is trained to maximize the similarity between different views of the same image, while minimizing the similarity between views of different images.

The Self-Supervised Learning (SSL) method proposed by DINO is also useful for medical imaging when class imbalance renders other SSL methods ineffective. In DINO, self-distillation is used to train a student network without labels by matching its output to the output of a teacher network across many views of the image. DINO uses exponential moving average (EMA) on the student weights, i.e., a momentum encoder, to update the teacher model. The deep learning model 314 passes two different random transformations of the input image to the student and the teacher networks. Both the student and the teacher networks have the same architecture but different parameters. The architecture of the instructor network is identical to the architecture of the student network, which was constructed using the EMA of student parameters. Backpropagation is utilized to update only the network parameters of students. The output of the teacher network is centered by calculating the batch mean statistics, i.e., subtracting the batch mean. The outputs of both models are divided by a temperature hyperparameter and then normalized using a temperature softmax, and their similarity is evaluated using cross-entropy loss. By adopting DINO, the models used in the embodiments herein n are able to converge faster when trained for downstream tasks, such as metastatic classification, and handle stain variations more effectively than when utilizing ImageNet weights.

In an example herein, the image compression unit 108 generates 2×N×N patches 316. The image compression unit 108 encodes and compresses each patch of the top 2×N×N patches with the highest tumour percentage separately and generates an 8×8×2048 feature block 318. The image compression unit 108 stores the 8×8×2048 feature blocks 318 in memory, or on disk, or a storage unit (not shown). The image compression step is not trained for metastasis classification. ResNeXt50 SSL weights are utilized primarily for patch-level feature extraction. The image compression unit 108 reduces the dimension of the image from 256×256×3 to 8×8×2048. The initial image passed is of the size 256×256×3 but the output has dimension of 8×8×2048 which is smaller than the original size. The image compression unit 108 removes irrelevant information and reduces the size of the image.

During training, translation augmentations, such as, but not limited to, random rotate and flip are used alongside color augmentations like random brightness, contrast, and color jitter. The image compression unit 108 implements Gaussian noise and pixel dropout.

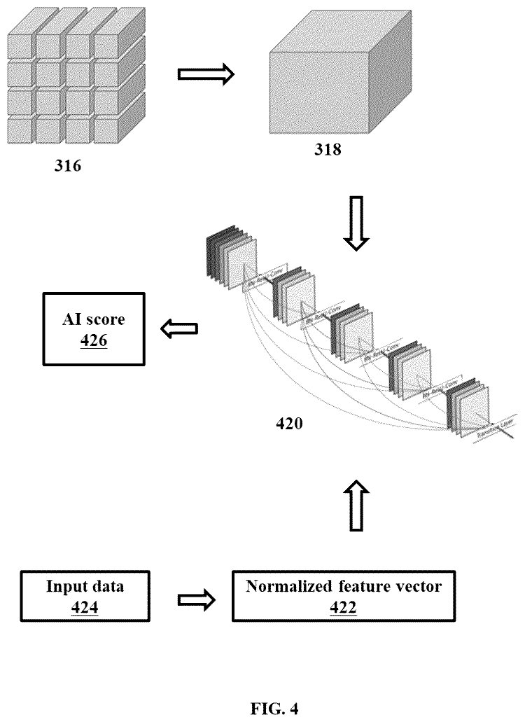

FIG. 4 illustrates the classification process 400 for predicting the risk of metastasis in subjects post radical prostatectomy, according to the embodiments herein. At each training cycle, the classification unit 110 selects N×N features at random from the 2×N×N patch representation generated during image compression. This random selection of training patches is an effective strategy for enhancing performance on tests. By randomly selecting N×N patches, a WSI can be represented in ^{2×N×N}C_{N×N} ways, hence increasing the variety of training data and making the model more resistant to overfitting. In addition, complexity of the training data is increased due to introduction of randomness and noise. Despite a modest training sample size, increasing both diversity and complexity during training increases the network's capacity to generalize.

The classification unit 110 forms a tensor of size 8N×8N×2048 by concatenating the features of N×N patch representations. The classification unit 110 orders features according to the proportion of tumours, i.e., the first block will have the representation of the patch with the highest proportion of tumour and organizes the patches column by column. During inference, the classification unit 110 selects the top N×N features from the patches with the greatest tumour percentage and concatenates to produce an 8N×8N×2048 tensor.

The classification unit 110 employs a classifier 420. In an embodiment herein, the classifier 420 can use a deep learning model, such as, ResNet18. It is within the scope of the embodiments herein to use other deep learning models such as, but not limited to, convolutional neural networks (CNNs) and their variants, such as 3D-CNNs or residual networks (ResNets) for the classification. ResNet18 is a deep neural network architecture that belongs to the ResNet family of models. The ResNet18 architecture comprises of 18 layers, including a convolutional layer, a max-pooling layer, and several residual blocks. The residual blocks are the key feature of ResNet18, and they allow the model to learn increasingly complex features from the input images. Each residual block in ResNet18 comprises of two convolutional layers and a shortcut connection that bypasses the convolutional layers. The shortcut connection allows the model to learn from the identity mapping of the input data and helps to mitigate the vanishing gradient problem that can occur in deep neural networks. The convolutional layers within each residual block use 3×3 filters, and the number of filters is increased gradually throughout the layers to allow the model to learn increasingly complex features. The last layer of ResNet18 is a fully connected layer that performs the final classification of the input image. ResNet18 is advantageous because of its relatively small size, which makes it computationally efficient and easy to train, while still achieving high accuracy on many tasks.

The classification unit 110 receives two inputs: one is the clinical data and the other one is the image data. In an example, as explained above, the ResNet18, which is the CNN part of the classification unit 110, processes the image data. A two-layer fully connected network, also known as the 2 layer MLP, processes the clinical data which is passed to it in vector form. In an example, the vector can be a 256-dimensional vector 422. The classification unit 110 concatenates this vector with ResNet18's penultimate layer output and the concatenated network is passed through a final layer. The output from the final layer is passed to softmax function to obtain an AI risk score for determining the risk of the metastasis.

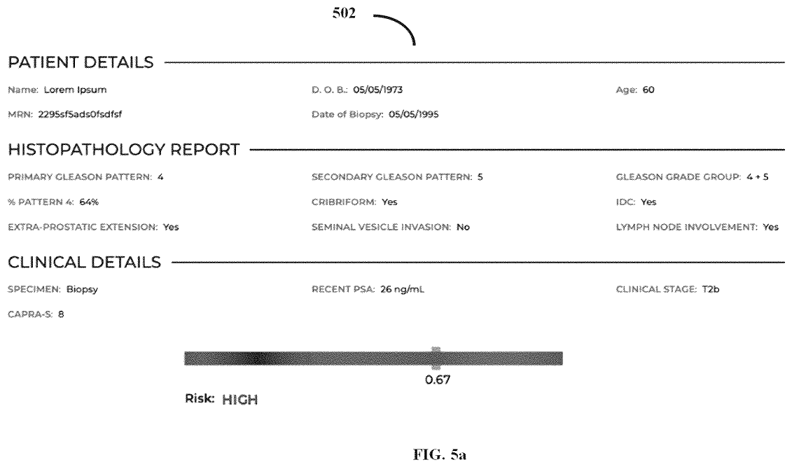

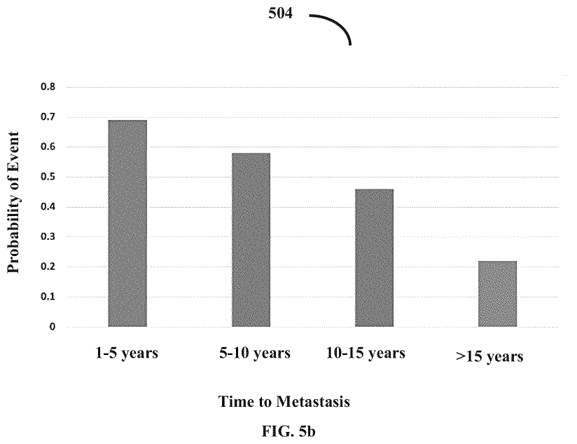

FIG. 5a illustrates a sample report 502 showing the risk of metastasis as determined by the concatenated feature based classification system, according to the embodiments herein. FIG. 5b illustrates a graph which depicts the probability of metastatic event at different time intervals 504, according to embodiments herein.

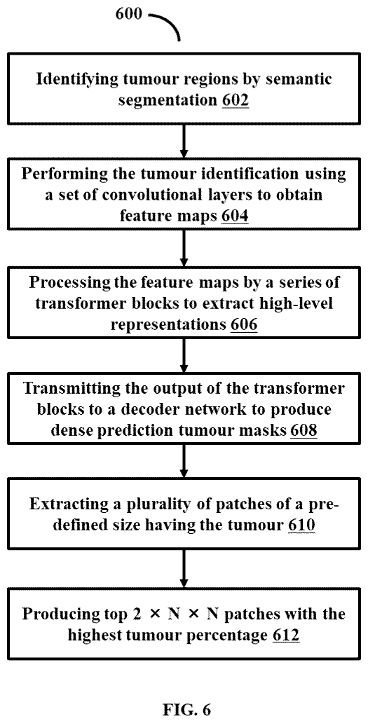

FIG. 6 depicts the process of tumour identification and patch generation, according to the embodiments herein. At step 602, the tumour identification and patch generation unit 106 identifies tumour regions by performing semantic segmentation using a semantic segmentation network, such as, but not limited to SegFormer or U-net. In semantic segmentation, each pixel in the input histopathological images is labelled with a semantic class. At step 604, the tumour identification and patch generation unit 106 identifies the tumour by first encoding the input histopathological images using a set of convolutional layers to obtain a set of feature maps. At step 606, the tumour identification and patch generation unit 106 processes these feature maps by a series of transformer blocks to extract high-level representations that capture both local and global contexts. At step 608, the tumour identification and patch generation unit 106 transmits the output of the transformer blocks to a decoder network that produces a dense prediction tumour masks for each pixel. At step 610, on producing dense prediction tumour masks for each pixel of the input images, the tumour identification and patch generation unit 106 performs patch generation which involves extracting at least one patch having at least a pre-defined amount of tumour from the identified tumour regions. In an example herein, the pre-defined amount can be 10% tumour in the identified tumour region. At step 612, the tumour identification and patch generation unit 106 produces top 2×N×N patches with the highest tumour percentage.

The various actions in method 600 may be performed in the order presented, in a different order or simultaneously. Further, in some embodiments, some actions listed in FIG. 600 may be omitted.

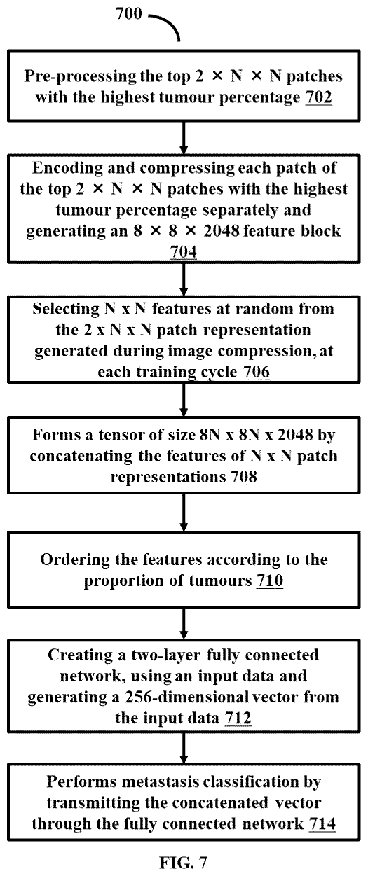

FIG. 7 depicts an example process of image compression and classification for predicting the risk of metastasis, according to the embodiments herein. At step 702, the image compression unit 108 preprocesses the top 2×N×N patches with the highest tumour percentage by using DINO approach. The pre-processing normalizes the size and intensity values of the patches. At step 704, the image compression unit 108 encodes and compresses each patch of the top 2×N×N patches with the highest tumour percentage separately and generates an 8×8×2048 feature block. At step 706, the classification unit 110 selects N×N features at random from the 2×N×N patch representation generated during image compression, at each training cycle. At step 708, the classification unit 110 forms a tensor of size 8N×8N×2048 by concatenating the features of N×N patch representations. At step 710, the classification unit 110 orders features according to the proportion of tumours, i.e., the first block will have the representation of the patch with the highest proportion of tumour and organizes the patches column by column. At step 712, the classification unit 110 creates a two-layer fully connected network, using an input data and generates a 256-dimensional vector from the input data. The input data can be, but not limited to, clinical data, histopathological images, molecular markers, and radiology data. At step 714, the classification unit 110 concatenates this vector with the 8N×8N×2048 tensor and performs metastasis classification by transmitting the concatenated vector through the fully connected network. The classification unit 110 generates an AI score to determine the risk of metastasis.

The various actions in method 700 may be performed in the order presented, in a different order or simultaneously. Further, in some embodiments, some actions listed in FIG. 700 may be omitted.

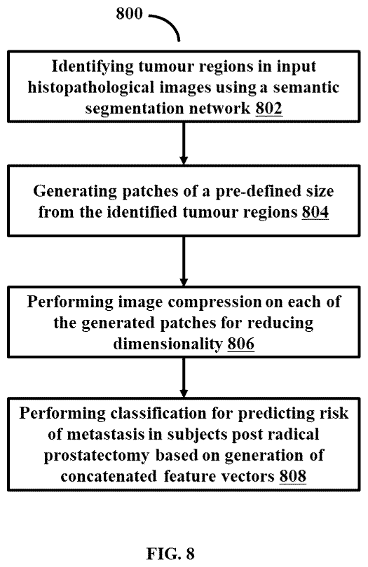

FIG. 8 depicts the process of predicting risk of metastasis, according to the embodiments herein. At step 802, the tumour identification and patch generation unit 106 identifies tumour regions in input histopathological images using a semantic segmentation network. At step 804, the tumour identification and patch generation unit 106 generates patches of a pre-defined size from the identified tumour regions. At step 806, the image compression unit 108 performs image compression on each of the generated patches for reducing dimensionality. At step 808, the classification unit 110 performs classification for predicting risk of metastasis in subjects post radical prostatectomy based on generation of concatenated feature vectors.

The various actions in method 800 may be performed in the order presented, in a different order or simultaneously. Further, in some embodiments, some actions listed in FIG. 800 may be omitted.

The embodiments disclosed herein can be implemented through at least one software program running on at least one hardware device and performing network management functions to control the network elements. The network elements shown in FIG. 1 include blocks which can be at least one of a hardware device, or a combination of hardware device and software module.

The embodiments disclosed herein describe a multi-modality deep learning system which can predict the likelihood of metastases in subjects post radical prostatectomy. The embodiments herein disclose employ the trained deep learning system utilizing multimodal data, including histopathology images and clinical data from a group of subjects with long-term follow-up, for predicting the risk of metastases post radical prostatectomy.

Therefore, it is understood that the scope of the protection is extended to such a program and in addition to a computer readable means having a message therein, such computer readable storage means contain program code means for implementation of one or more steps of the method, when the program runs on a server or mobile device or any suitable programmable device. The method is implemented in at least one embodiment through or together with a software program written in e.g. Very high speed integrated circuit Hardware Description Language (VHDL) another programming language, or implemented by one or more VHDL or several software modules being executed on at least one hardware device. The hardware device can be any kind of portable device that can be programmed. The device may also include means which could be e.g. hardware means like e.g. an ASIC, or a combination of hardware and software means, e.g. an ASIC and an FPGA, or at least one microprocessor and at least one memory with software modules located therein. The method embodiments described herein could be implemented partly in hardware and partly in software. Alternatively, the invention may be implemented on different hardware devices, e.g. using a plurality of CPUs.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of embodiments and examples, those skilled in the art will recognize that the embodiments and examples disclosed herein can be practiced with modification within the spirit and scope of the embodiments as described herein.

1. A method for predicting risk of metastasis in post radical prostatectomy of a patient, the method comprising:
identifying, by a tumour identification and patch generation unit, at least one tumour region in an input histopathological image of the post radical prostatectomy of the patient, using a semantic segmentation network;
generating, by the tumour identification and patch generation unit, at least one patch of a pre-defined size from the at least one identified tumour region;
performing, by an image compression unit, image compression on the at least one patch to reduce dimensionality;
performing, by a classification unit, classification of input data to predict risk of metastasis in the subjects post radical prostatectomy, wherein the classification is based on generation of concatenated feature vectors at training stage; and
generating an AI score based on the classification of the input data, wherein the AI score indicates the risk of metastasis in post radical prostatectomy of the patient.

2. The method as claimed in claim 1, wherein identifying, by a tumour identification and patch generation unit, tumour regions in input histopathological images, using a semantic segmentation network, comprises:
performing the tumour identification by first encoding the input histopathological images using a set of convolutional layers to obtain a set of feature maps;
processing the set of feature maps by a series of transformer blocks to extract high-level representations that capture both local and global context; and
transmitting an output of the series of transformer blocks to a decoder network that produces a dense prediction tumour masks for each pixel of the input histopathological images.

3. The method, as claimed in claim 1, wherein generating, by the tumour identification and patch generation unit, the at least one patch of a pre-defined size from the identified tumour regions, comprises:
performing, by the tumour identification and patch generation unit, patch generation of the dense prediction tumour masks for the each pixel of the input histopathological images, wherein the patch generation comprises extracting the at least one patch having at least 10% tumour from the identified tumour regions; and
producing, by the tumour identification and patch generation unit, top 2×N×N patches with the highest tumour percentage.

4. The method, as claimed in claim 1, wherein performing, by the image compression unit, the image compression on the at least one patch comprises:
pre-processing the top 2×N×N patches with the highest tumour percentage by using DINO approach, wherein the pre-processing normalizes size and intensity of values of the at least one patch; and
encoding and compressing each of the patch of the top 2×N×N patches with the highest tumour percentage separately and generating an 8×8×2048 feature block.

5. The method, as claimed in claim 1, wherein classification of the input data by the classification unit, for predicting the risk of metastasis in subjects post radical prostatectomy, comprises:
selecting N×N features at random from the top 2×N×N patch generated during the image compression, at each training cycle;
forming a tensor of size 8N×8N×2048 by concatenating patch representations of the N×N features;
creating a two-layer fully connected network, using an input data and generating a 256-dimensional vector from the input data;
concatenating the 256-dimensional vector with the 8N×8N×2048 tensor; performing metastasis classification by transmitting the concatenated vector through the two-layer fully connected network; and
generating an AI score to determine the risk of metastasis.

6. A concatenated feature based system for predicting risk of metastasis in post radical prostatectomy of a patient, the system comprising:
a computing device communicatively coupled with a data repository, wherein the computing device comprises a plurality of units comprises a tumour identification and patch generation unit, an image compression unit, and a classification unit,
wherein the tumour identification and patch generation unit is to:
identify at least one tumour region in an input histopathological image of the post radical prostatectomy of the patient, using a semantic segmentation network; and
generate at least one patch of a pre-defined size from the at least one identified tumour region;
wherein the image compression unit is to perform image compression on at least one patch to reduce dimensionality;
wherein the classification unit is to perform classification of input data to predict risk of metastasis in the subjects post radical prostatectomy, wherein the classification is based on generation of concatenated feature vectors at training stage and to generate an AI score based on the classification of the input data, wherein the AI score indicates the risk of metastasis in the post radical prostatectomy of the patient.

7. The system as claimed in claim 6, wherein the tumour identification and patch generation unit identifies tumour regions in input histopathological images, using a semantic segmentation network, by:
performing the tumour identification by first encoding the input histopathological images using a set of convolutional layers to obtain a set of feature maps;
processing the set of feature maps by a series of transformer blocks to extract high-level representations that capture both local and global context; and
transmitting an output of the series of transformer blocks to a decoder network that produces a dense prediction tumour masks for each pixel of the input histopathological images.

8. The system, as claimed in claim 6, wherein the tumour identification and patch generation unit generates the at least one patch of the pre-defined size from the identified tumour regions, by:
performing patch generation of the dense prediction tumour masks for the each pixel of the input histopathological images, wherein the patch generation comprises extracting the at least one patch having at least 10% tumour from the identified tumour regions; and
producing top 2×N×N patches with the highest tumour percentage.

9. The system, as claimed in claim 6, wherein the image compression unit performs the image compression on the at least one patch by:
pre-processing the top 2×N×N patches with the highest tumour percentage by using DINO approach, wherein the pre-processing normalizes size and intensity of values of the at least one patch; and
encoding and compressing each of the patch of the top 2×N×N patches with the highest tumour percentage separately and generating an 8×8×2048 feature block.

10. The system, as claimed in claim 6, wherein the classification unit classifies the input data for predicting the risk of metastasis in subjects post radical prostatectomy, by:
selecting N×N features at random from the top 2×N×N patch generated during the image compression, at each training cycle;
forming a tensor of size 8N×8N×2048 by concatenating patch representations of the N×N features;
creating a two-layer fully connected network, using an input data and generating a 256-dimensional vector from the input data;
concatenating the 256-dimensional vector with the 8N×8N×2048 tensor; performing metastasis classification by transmitting the concatenated vector through the two-layer fully connected network; and
generating an AI score to determine the risk of metastasis.
